(19) United States
(12) Patent Application Publication
(10) Pub. No.: US 2022/0355090 A1
(43) Pub. Date: Nov. 10, 2022

(54) Drive device for a hand-held device for locally puncturing a skin, and hand-held device

(71) Applicant: MT.DERM GmbH, Berlin (DE)

(72) Inventor: Dirk Scherkowski, Berlin (DE)

(21) Appl. No.: 17/737,167

(22) Filed: May 5, 2022

(30) Foreign Application Priority Data
May 5, 2021 (EP) 21172237.6

(51) Int. Cl. A61M 37/00; A01K 11/00

(52) U.S. Cl. CPC A61M 37/0076; A01K 11/005; A61M 2205/103

(57) ABSTRACT

The invention relates to a drive device for a hand-held device (1) for locally puncturing a skin, having a housing (5) which is connectable to a module housing (8) of a skin-puncturing device; and an electrical drive device which is arranged in the housing (5) and has a tapping component which is designed to provide a tap for tapping a rotary drive movement generated by the electric drive device for the skin-puncturing device. The electric drive device has an electric motor designed as an external rotor with a rotating rotor component, in which the tapping component is arranged on the rotor component and eccentrically in relation to a rotor axis. Furthermore, a hand-held device (1) for locally puncturing a skin is provided.

The invention relates to a drive device for a hand-held device for locally puncturing a skin, and a hand-held device for locally puncturing a skin.

### BACKGROUND

Such hand-held devices are used to locally puncture human or animal skin, in particular in connection with tattooing or for the formation of permanent makeup. In this case, a dye is introduced into the skin during and/or after the local puncturing of the skin. However, local puncturing of the skin with such a hand-held device can also be used to introduce cosmetic or medical active ingredients. Alternatively, the use of such hand-held devices for locally puncturing the skin without introducing any substance or active ingredient is known, for example, for skin stimulation.

In the case of hand-held devices for locally puncturing the skin, a drive device with an electric motor is usually provided, with which a rotary drive movement is provided. The drive movement is tapped by means of a coupling device and converted into a linear movement, which is coupled to a puncturing device in order to move one or more puncturing needles forward and backward during operation at a repetition frequency in order to locally puncture the skin.

### SUMMARY

The problem addressed by the invention is that of providing a drive device for a hand-held device for locally puncturing a skin, as well as such a hand-held device with which a drive movement for a puncturing device can be provided efficiently and flexibly for different applications.

The problem is solved by a drive device for a hand-held device for locally puncturing a skin and by a hand-held device for locally puncturing a skin according to independent claims 1 and 12. Embodiments are the subject matter of dependent claims.

According to one aspect, a drive device for a hand-held device for locally puncturing a skin is provided, which has the following: a housing which is connectable to a module housing of a skin-puncturing device; and an electric drive device which is arranged in the housing and has a tapping component which is designed to provide a tap for tapping a rotary drive movement generated by means of the electric drive device for the skin-puncturing device. The electric drive device has an electric motor designed as an external rotor (outrunner) with a rotating rotor component, in which the tapping component is arranged on the rotor component and eccentrically in relation to a rotor axis.

According to a further aspect, a hand-held device for locally puncturing a skin having a drive device and a skin-puncturing device having a module housing is provided, which is detachably connected to the drive device and in which, during operation, a puncturing device can be moved forward and backward in a module housing opening on the front. The hand-held device comprises a coupling device which is formed as part of the drive device and/or the skin-puncturing device and is designed to tap a rotary drive movement provided in the drive device at the tap of an electric motor designed as an external rotor (outrunner) and to couple it to a linear movement for the puncturing device.

It is provided that the tap is provided on the electric motor separately from the motor shaft on the rotor component of the electric motor. It is thus prevented that additional components, on which the tap is subsequently formed, have to be mounted on the motor shaft to form an eccentricity of the tap in relation to the rotor or motor axis of the electric motor. In this way, the drive device can be designed with improved mechanical stability.

By arranging the tap on the rotor component of the external rotor, the encasing of a motor shaft by a usually cylindrical, eccentrically arranged tapping component on the motor shaft, which is otherwise necessary in the prior art, is no longer required. The tapping component arranged on the rotor component (not on the motor shaft) can thus be selected to be smaller in terms of its dimensions, optionally up to a size smaller than the motor shaft, for example, with regard to a diameter. The tap provided on the rotor component itself is stressed solely by a bending moment and can therefore be kept smaller.

A conversion mechanism is provided or implemented with the coupling device, with which the rotary drive movement is converted into the linear movement, as is known as such for hand-held devices in various configurations for locally puncturing a skin. The coupling of the drive movement to the puncturing device can be designed such that the puncturing device is forced to move forward and/or backward as a result. Alternatively or additionally, other or further forces can be coupled to the puncturing device, which cause or support its movement when moving forward and/or backward, for example, a spring force and/or a force applied by a stretched membrane, as is also known as such in various designs.

By forming the tapping component on the rotor component itself, an eccentric crank pin can be provided, on which the rotary drive movement can be tapped. In one embodiment, the rotor component, on which the tapping component is arranged, can be a rotating outer bell or a rotating housing component of the electric motor.

It can be provided for the electric motor to be designed without a motor shaft coupling pin, since the rotary drive movement is tapped via the tapping component on the rotor component. The electric motor can thus be free of an external motor shaft.

The tapping component can be arranged on an end face of the rotor component. Alternatively, the tapping component can be arranged in the region of a circumferential surface of the rotor component or in the region of a transition between the end face and the circumferential surface.

The tapping component can be movably arranged on the rotor component such that an eccentricity of the tap in relation to the rotor axis is adjustable. The movability of the tapping component on the rotor component is provided in particular in the radial direction, so that a distance between the tapping component providing the tap and the rotor or motor axis can be increased and decreased. For this purpose, the tapping component can be mounted on the rotor component, for example, by means of a detachable clamp or screw connection which can be loosened in order to move the tapping component on the rotor component. After moving, the connection can be resecured to fix the tapping component on the rotor component in the new position. By means of a change in the eccentricity, a stroke for the puncturing device (length of the forward and backward movement; maximum distance between reversal points of a linear puncturing movement) can be set when coupling the drive device to the skin-puncturing device, in particular a needle stroke of a needle device of the skin-puncturing device having one or more puncturing needles which are moved forward and backward for locally puncturing the skin.

The tapping component can be integrally formed on the rotor component. For the integral forming of the tapping component on the rotor component, it can be provided that, for example, an adhesive or welded connection is formed between the components.

The tapping component can be detachably arranged on the rotor component. A detachable connection between the tapping component and the rotor component can be formed, for example, by means of a screw connection or a plug connection.

A balance mass can be assigned to the tapping component. Vibrations during operation of the drive device are reduced or completely eliminated by means of the balance mass assigned to the tapping component.

The balance mass can be formed with a balance component which is mounted or arranged on the rotor component. The balance component can be movably arranged on the rotor component. In this way, an adjustability of the balancing or compensating effect is made possible, for example, when the eccentricity for the tap on the rotor component is changed.

The balance component can be arranged on the tapping component. In this embodiment, the balance component is mounted on the tapping component. For example, a projection or a pin of the tapping component can pass through an opening on the balance component if it is mounted on the tapping component. In this case, it can be provided that the balance component can be fixed in several rotational positions around the tapping component, for example, by means of a detachable locking screw, in order to provide the balancing effect in a changeable manner.

A bearing component can be arranged on the tapping component, which is designed to be coupled to a connecting rod component for tapping the rotary drive movement. The mounting of the connecting rod component on the tapping component can be carried out, for example, using a ball bearing or a plain bearing.

The tapping component can be arranged on a rotor housing component. For example, the rotor housing component can be a rotating outer bell of the electric motor. The rotor housing component can be cup-, ring- or disc-shaped.

A handpiece that can be gripped by a user can be formed with the housing of the drive device.

In the case of the hand-held device for locally puncturing a skin, the skin-puncturing device can be formed with a puncturing module designed as a disposable module. Disposable modules designed as a puncturing module are known as such in different embodiments. They make it possible to replace the skin-puncturing device after use in order to provide a hand-held device with a high standard of hygiene.

In the case of the hand-held device for local puncturing, the coupling device can have a connecting rod component which is connected in a rotationally decoupling manner to a tapping component of the electric motor providing the tap. The connecting rod component can be designed with a connecting rod which, on the drive side, has a connecting rod eye which, in a coupled position, partially or essentially completely encompasses the tapping component. A rotational decoupling can be achieved by means of a ball bearing or a plain bearing. If a balance component is arranged on the tapping component to provide a balance mass assigned to the tapping component, the balance component can be arranged in a proximal position with respect to the rotor component, whereas a bearing of the connecting rod component is formed on the tapping component in a distal position.

The further configurations described above in connection with the drive device can be provided accordingly in connection with the hand-held device for locally puncturing the skin.

### DESCRIPTION OF EMBODIMENTS

Further embodiments are explained below with reference to figures in the drawings, in which:

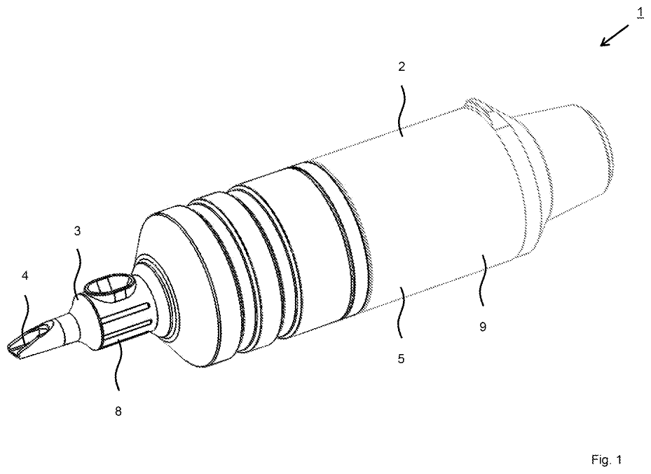

FIG. 1 shows a schematic perspective depiction of a hand-held device for locally puncturing a skin, having a drive device and a skin-puncturing device connected thereto, which are formed with a drive module and a needle module;

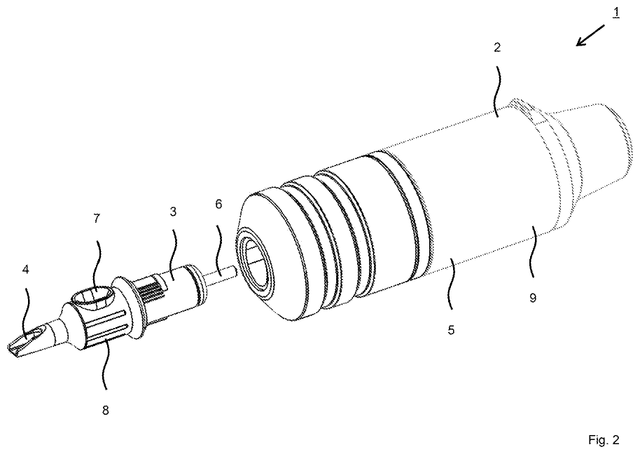

FIG. 2 shows a schematic perspective depiction of the hand-held device from FIG. 1, wherein the skin-puncturing device formed with the needle module is separated from the drive device;

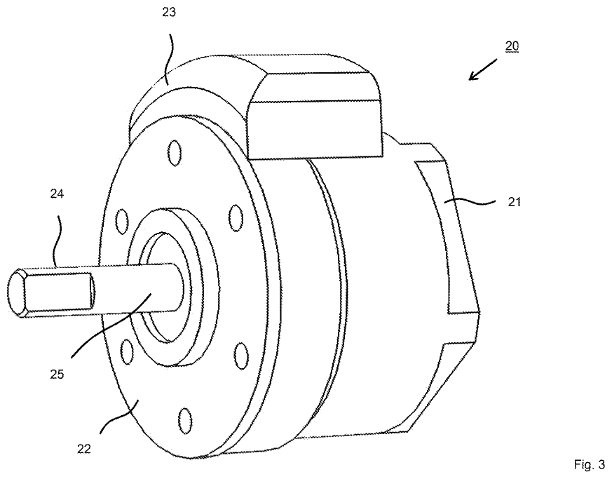

FIG. 3 shows, diagonally from the front, a perspective depiction of an electric motor designed as an external rotor;

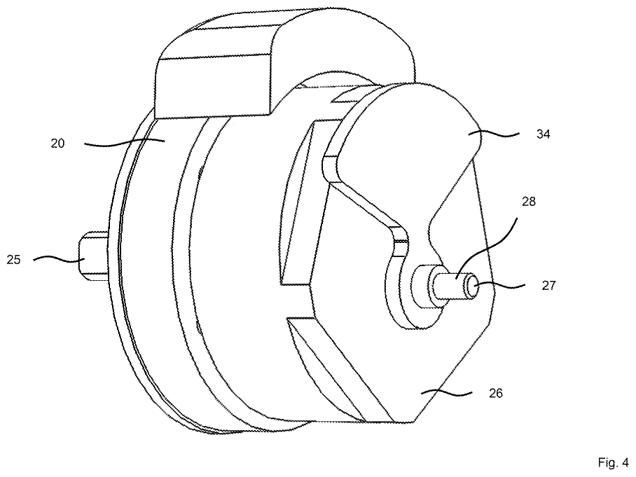

FIG. 4 shows, diagonally from the rear, a schematic perspective depiction of the electric motor from FIG. 3;

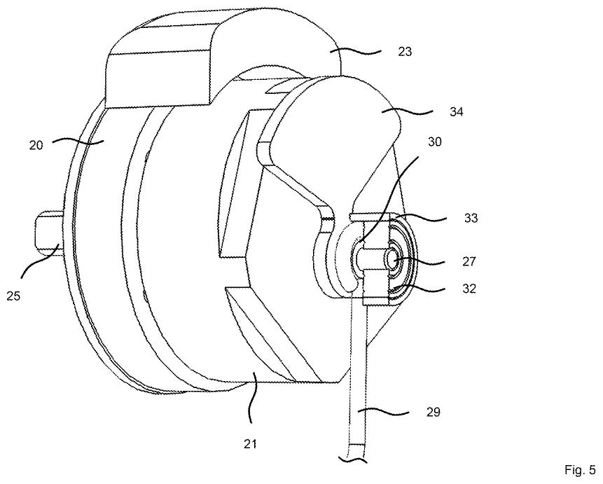

FIG. 5 shows, diagonally from the rear and partially in section, a schematic perspective depiction of the electric motor from FIG. 4, wherein a connecting rod component is coupled to an eccentric tap;

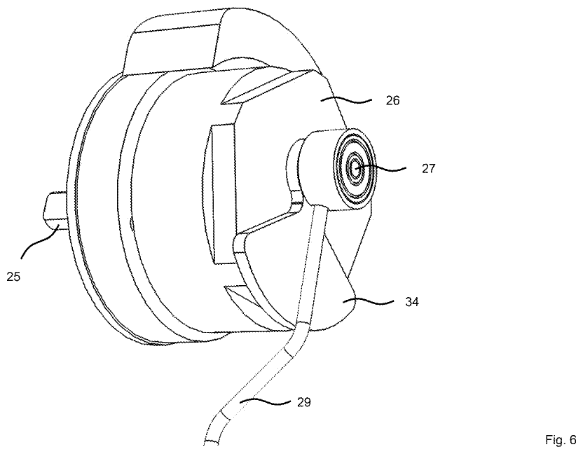

FIG. 6 shows, diagonally from the rear, a schematic perspective depiction of the electric motor from FIG. 5 in a different rotational position;

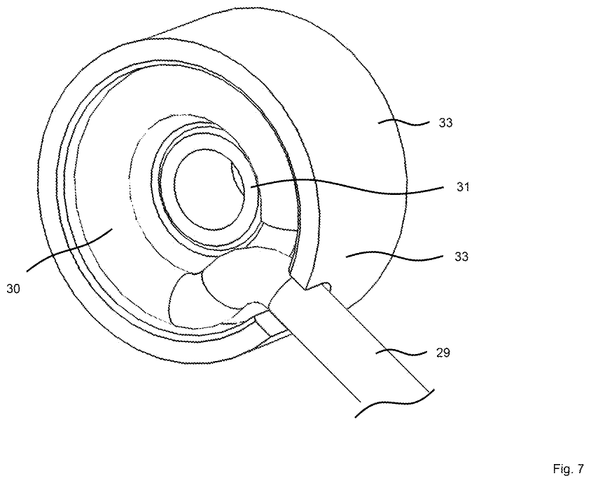

FIG. 7 shows a schematic perspective depiction of an end portion of the connecting rod component in detail;

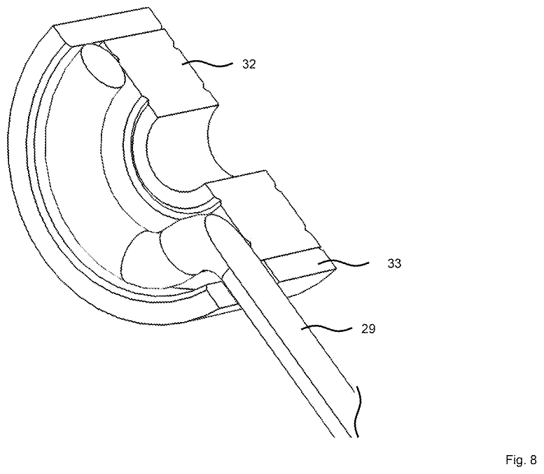

FIG. 8 shows a schematic perspective depiction of the end portion of the connecting rod component from FIG. 7 in detail and in section;

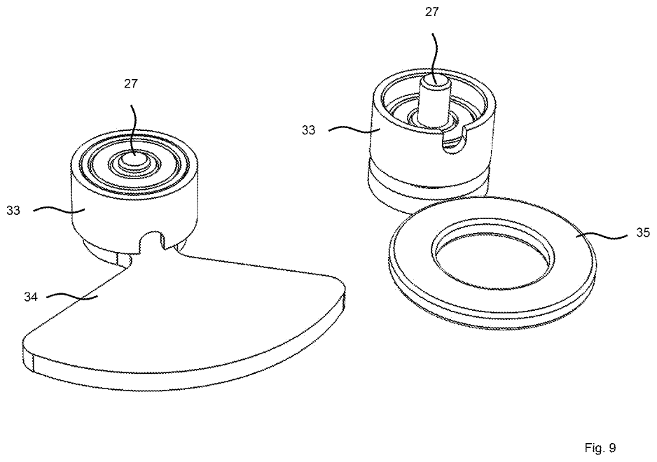

FIG. 9 shows a schematic perspective depiction of a balance component with one-piece and multi-piece design.

FIGS. 1 and 2 show schematic perspective depictions of a hand-held device 1 having a drive module 2 providing a drive device and a needle module 3 coupled thereto and providing a skin-puncturing device, which can be detached from the hand-held module 2 according to FIG. 2, in particular after the hand-held device 1 has been used for locally puncturing a skin in order to dispose of the needle module 3 which can be designed as a disposable module.

The hand-held device 1 is designed to puncture human or animal skin several times locally during operation with a repetition frequency. For this purpose, a puncturing device having one or more puncturing needles (not shown) is moved forward and backward through a housing opening 4 on the front on the needle module 3, as it is known per se. In order to provide the linear forward and backward movement of the puncturing device, a coupling device (not shown) is provided, which, in the example shown, is arranged in a housing 5 of the drive module 2 and designed to tap a rotary drive movement provided by an electric motor of the drive device and convert it into a linear movement for moving the puncturing device forward and backward and to couple it directly or indirectly to the one or more puncturing needles. A conversion mechanism is thus provided with the coupling device, as is known per se for such hand-held devices. The puncturing device is then moved forward and backward at a repetition frequency.

The linear drive movement is transmitted to the puncturing device via a coupling component 6 (cf. FIG. 2) of the skin-puncturing device formed with the needle module 3, which is connected to the one or more puncturing needles of the puncturing device, so that the one or more puncturing needles can be moved forward and backward. The coupling component 6 can be part of the coupling device or formed separately therefrom in order to tap the linear movement and transfer it to the puncturing device.

The hand-held device 1 can be designed as a tattooing device or as a device for creating permanent makeup. In this case, a dye to be introduced into the skin can be supplied via an opening 7 on a module housing 8. However, the hand-held device 1 can also be used for locally puncturing the skin without introducing any substance.

A handpiece 9 is formed with the housing 5 of the drive device provided by means of the drive module 2 and can be gripped by the user in order to guide the hand-held device 1 during operation.

In the following and with reference to FIGS. 3 to 9, embodiments of an electric motor 20 designed as an external rotor (outrunner) are described, which is accommodated in the housing 5 of the drive device provided by means of the drive module 2 in order to provide the rotary drive movement. In the case of the external rotor, an external rotor component 21 rotates during operation. In the embodiment shown, the rotor component 21 forms a rotating outer bell or a rotating outer housing of the electric motor 20, which can be cup-shaped. The electric motor 20 can be mounted using a docking flange 22. Electrical connections/taps are arranged in a block 23.

The rotor component 21 rotates about a motor or rotor axis formed in the axial direction of a motor shaft 24. A protruding part 25 of the motor shaft 24 can be omitted.

For the operation of the hand-held device 1, it is provided that an eccentric rotary movement is provided by means of the electric motor 20, which forms a drive movement for moving the puncturing device forward and backward. For this purpose, according to FIG. 4, a tapping component 27 eccentrically arranged in relation to the motor shaft 24 is provided on a (rear) end face 26 of the rotor component 21 and formed with an eccentric crank pin 28 in the embodiment. The tapping component 27 can be, for example, welded or glued to the rotor component 21. A detachable mounting on the rotor component 21 can be provided in particular such that the tapping component 27 can be fixed in several positions at different distances from the motor shaft 24 in order to adjust the eccentricity.

In order to eccentrically tap the rotary drive movement, the tapping component 27 is coupled to a connecting rod component 29 (cf. FIGS. 5 and 6). The end of the connecting rod component 29 is formed with a connecting rod eye 30 which, in the example shown, essentially completely encompasses a shaft shoulder 30 on the crank pin 28. The connecting rod component 29 is connected to the tapping component 27 in a rotationally decoupled manner. For this purpose, a ball bearing 32 is provided in a surrounding outer ring 33, which is arranged on the eccentrically arranged crank pin 28 above the connecting rod component 29.

In particular according to FIGS. 4 to 6, a balance component 34 is also provided on the tapping component 27, which provides a balance mass assigned to the tapping component 27 for balance. For the balance component 34, FIGS. 7 to 9 show the one-piece design and an alternative multi-piece design. In the case of the multi-part design, a standard component 35 can be glued or welded to the outer side of the rotor component 21.

It can be provided that the balance component is movably arranged on the rotor component 21 such that a distance between a rotor axis of the electric motor 20 and the tapping component is changeable in the radial direction, so that the eccentricity of the tapping component is adjustable. By interacting with the connecting rod component, a stroke for the forward and backward movement of the puncturing device can thus be adjusted.

The features disclosed in the above description, the claims, and the drawings can be of relevance, both individually and also in any combination, for realizing the different embodiments.

1. A drive device for a hand-held device for locally puncturing a skin, comprising:
a housing which is connectable to a module housing of a skin-puncturing device; and
an electrical drive device which is arranged in the housing and has a tapping component configured to provide a tap for tapping a rotary drive movement generated by the electric drive device for the skin-puncturing device;
wherein the electric drive device has an electric motor designed as an external rotor with a rotating rotor component, wherein the tapping component is eccentrically arranged on the rotor component in relation to a rotor axis.

2. The drive device according to claim 1, wherein the tapping component is arranged on an end face of the rotor component.

3. The drive device according to claim 1, wherein the tapping component is movably arranged on the rotor component such that an eccentricity of the tap in relation to the rotor axis is adjustable.

4. The drive device according to claim 1, wherein the tapping component is integrally formed on the rotor component.

5. The drive device according to claim 1, wherein the tapping component is detachably arranged on the rotor component.

6. The drive device according to claim 1, wherein a balance mass is assigned to the tapping component.

7. The drive device according to claim 6, wherein the balance mass is formed with a balance component which is mounted on the rotor component.

8. The drive device according to claim 7, wherein the balance component is arranged on the tapping component.

9. The drive device according to claim 1, further comprising a bearing component arranged on the tapping component, the bearing component being designed to couple to a connecting rod component for tapping the rotary drive movement.

10. The drive device according to claim 1, wherein the tapping component is arranged on a rotor housing component.

11. The drive device according to claim 1, wherein a handpiece is formed with the housing.

12. A hand-held device for locally puncturing a skin, comprising:
a drive device according to claim 1;
a skin-puncturing device having a module housing which is detachably connected to the drive device and in which, during operation, a puncturing device is connected for forward and backward movement in a module housing opening on the front; and
a coupling device which is formed with the drive device and/or the skin-puncturing device and is designed to tap a rotary drive movement provided in the drive device at the tap of an electric motor designed as an external rotor and convert it into a linear movement and couple said linear movement to the puncturing device.

13. The hand-held device according to claim 12, wherein the skin-puncturing device is formed with a needle module designed as a disposable module.

14. The hand-held device according to claim 12, wherein the coupling device has a connecting rod component which is connected in a rotationally decoupling manner to a tapping component of the electric motor providing the tap.

15. The hand-held device according to claim 12, further comprising a handpiece which is grippable by a user and which is formed with a housing of the drive device.
